(19) United States
(12) Patent Application Publication
(10) Pub. No.: US 2023/0102841 A1
(43) Pub. Date: Mar. 30, 2023

(54) FIXATION SUPPORT DEVICE FOR BRACHYTHERAPY WITH OR WITHOUT IMAGE GUIDANCE

(71) Applicant: COWLES VENTURES, LLC, Spokane, WA (US)

(72) Inventors: Emily Lin, Spokane, WA (US); Rodrigo Umanzor, Spokane, WA (US); Christopher Porter, Spokane, WA (US); Kevin Kelley, Spokane, WA (US); Mary Ann Slavik, Spokane, WA (US)

(73) Assignee: COWLES VENTURES, LLC, Spokane, WA (US)

(21) Appl. No.: 18/075,995

(22) Filed: Dec. 6, 2022

Related U.S. Application Data
(63) Continuation of application No. PCT/US2021/036474, filed on Jun. 8, 2021.
(60) Provisional application No. 63/036,266, filed on Jun. 8, 2020.
(60) Provisional application No. 63/088,280, filed on Oct. 6, 2020.

(51) Int. Cl. A61B 90/57; A61N 5/10

(52) U.S. Cl. CPC A61B 90/57; A61N 5/1001; A61B 2090/571

(57) ABSTRACT

A fixation support device may be used for breast brachytherapy treatment methods with or without image guidance. The fixation device may also be used for other treatments in which it is desirable to be able to fixate a treatment modality relative to a patient.

### RELATED APPLICATIONS

This application is a continuation of PCT/US2021/036474 filed Jun. 8, 2021 that in turn claims priority under 35 USC 119(e) and claims the benefit of U.S. Provisional Application No. 63/036,266, filed Jun. 8, 2020 and U.S. Provisional Application No. 63/088,280, filed Oct. 6, 2020, all of which are both incorporated herein by reference.

### FIELD

The disclosure relates generally to a mechanism and method for radiation oncology.

### BACKGROUND

When women are treated for breast cancer, the most commonly diagnosed cancer in women, they can opt for mastectomy (complete removal of the breast tissue) or breast conservation therapy (BCT) which is comprised of lumpectomy plus radiotherapy. Due to the widespread use of screening mammography, women are diagnosed with localized and early-stage disease so that BCT may be used. The typical radiation treatment is adjuvant breast radiation. While adjuvant breast radiation results in excellent survival rates, this regimen typically takes 3.5 to 7 weeks and is burdensome to women and the overall healthcare system. In addition, since the adjuvant breast radiation treatment is typically provided using external beam radiation, there is a greater risk of acute and late toxicity due to unnecessary healthy tissue interaction with the radiation.

As a result, accelerated partial breast irradiation (APBI) may be used which results in a quicker treatment time and less radiation-induced acute and late toxicity. One technique used for the APBI is brachytherapy. In one method, radioactive sources are temporarily or permanently implanted into the breast tissue at the site of the surgery wherein the radioactive sources may be high dose-rate or low dose-rate.

There are a handful of ways to insert radioactive sources into breast tissue. One is by a free hand method, another uses a compressive template device to temporary hold insertion catheters and the last uses a locking template system and non-fixated fiducial needle. These methods are limited in that they do not ensure the sources are placed in the desired location as prescribed by the treatment plan 100% of the time. The lack of ability to place the radioactive sources in the desired location means that the remaining tumor margin is not receiving the appropriate radiation and healthy tissue is receiving unwanted radiation.

In the high dose-rate brachytherapy area, a clinician would place hollow catheters into the breast to facilitate the insertion of a temporary radioactive source per a treatment plan which are then removed once the treatment is completed. The placement of these catheters may be by either free hand directly into the breast or by free hand though compressive template systems used to stereo-tactically immobilize the breast. Both Varian Medical Systems and Nucletron offer commercially available template immobilization products.

In the low dose-rate brachytherapy area, one method for permanent breast radioactive seed implantation is described in detail in “First Report of a Permanent Breast 103PD Seed Implant As Adjuvant Radiation Treatment for Early-Stage Breast Cancer”, Dr. Jean-Philippe Pignol et al., International Journal of Radiation Oncology Biological Physics, Vol. 64, No. 1, pp. 176-181 (2006) which is incorporated herein by reference. This method uses a non-fixated fiducial needle, locking template and stereotactic fixation to insert low dose rate (LDR) radioactive source strands into the treatment site under image guidance. In this method, the stereotactic fixation device uses a rail clamp to mount to a fixed frame of reference. For certain imaging modalities, the stereotactic fixation device does not meet the technical specifications of the imaging modality. For instance, a CT system does not have rails and certain materials can cause imaging artifacts. Thus, the system, a brachytherapy device, is limited for use in environments with tables that have side rails (such as an operating room) and with certain types of image guidance.

Thus, it is desirable to provide a device that can provide a fixed frame of reference for the system with the goal of expanding the use of the system in other environments and imaging modalities. It is to this end that the disclosure is directed.

It is noted that the following disclosure is particularly applicable to radioactive source implantation into breast tissue and it is in this context that the disclosure will be described. It will be appreciated, however, that the device and method have greater utility since the device can be used with other devices besides the template, needles, and radioactive sources and in various different tissues.

### BRIEF DESCRIPTION OF THE DRAWINGS

FIGS. 1A and 1B shows a fixation device positioned on the floor and the device located adjacent to a patient table.

FIG. 2 illustrates a method of using the device with a brachytherapy device.

FIGS. 3A and 3B illustrates another example of a device that is positioned on top of the table and the device on the table with a patient.

FIG. 4 illustrates another method of using the device in FIGS. 3A and 3B with a brachytherapy device.

FIGS. 5A and 5B are a top view and a bottom view of another example of a device.

FIG. 6 the fixation device of a CT table with a patient.

FIG. 7 illustrates another method of using the device with a CT System.

FIGS. 8A and 8B are a top view and a bottom view of another example of a device with handles that have recessed pockets on the bottom side.

FIGS. 9A and 9B are a top view and a bottom view of another example of a device with a rail on opposite sides.

### DETAILED DESCRIPTION OF ONE OR MORE EMBODIMENTS

The disclosure described allows the fixation of a brachytherapy device and can be implemented using the various devices and mechanisms described below. In one embodiment, the fixation device may be used for a breast brachytherapy treatment and it is in this context that the disclosure is provided, but it is understood that the fixation support device may be used for other treatments.

The fixation support device disclosed below is a portable device that is capable of stabilizing and fixating one or more medical modalities, such as one or more breast brachytherapy devices, with an attachment mechanism at a position on or near a treatment surface wherein the fixation support device is compatible with image guidance that may be used during the treatment. The fixation support device can have attachment mechanisms such as a rail clamp, pin, tightening knob, etc.

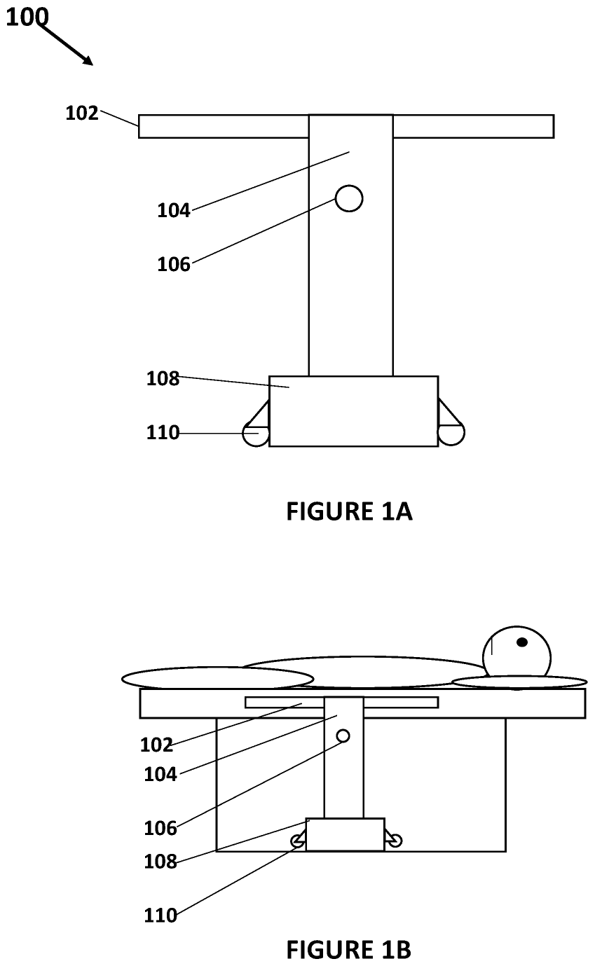

FIGS. 1A and 1B shows a fixation device 100 positioned on the floor and the device located adjacent to a patient table. The fixation device fixates a treatment modalities, such as a breast brachytherapy needle or template, relative to a fixed frame of reference in instances in which the treatment modalities could not otherwise fixated. For example, when a patient surgical table or bed lacks a rail that can be used, the fixation device 100 can be used. As shown in FIGS. 1A and 1B, the device 100 has a rail 102 to which the treatment modalities may be fixed that is attached to a vertical member 104 that holds the rail 102 a predetermined distance about a floor. The vertical member 104 has a height adjustment device 106, such as a knob, with a locking mechanism that allows the height of the rail 102 above the floor to be adjusted and then locked into position. The device 100 further has a base portion 108 at an end of the vertical member 104 opposite of the rail that has a set of wheels 110 that allow the device 100 to be rolled around on the floor or underneath/adjacent a patient bed/table as shown in FIG. 1B. The set of wheels 110 may be retractable so that the base portion 108 rests of the ground once it is appropriately positioned adjacent the table/bed as shown in FIG. 1B. Once in position, the rail 102 allows treatment elements, such as a brachytherapy template to be attached to the rail and locked relative to a fixed frame of reference.

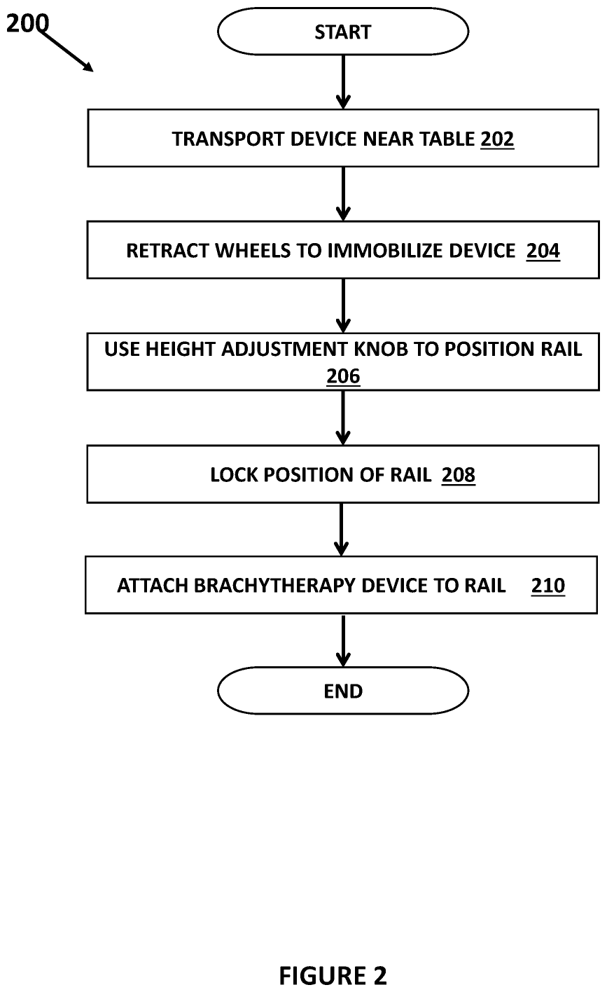

FIG. 2 illustrates a method 200 of using the device with a brachytherapy device. The device may be placed near a treatment surface (202) using the wheels. Once the device is appropriately positioned, the wheels are retracted off the floor (204) so that the device is immobilized. A height adjustment knob is used to position the rail near the treatment area (206) and then the device and thus the rail are locked in place (208). A brachytherapy device is attached to the rail (210) so that the clinician can perform the treatment procedure, such as breast brachytherapy.

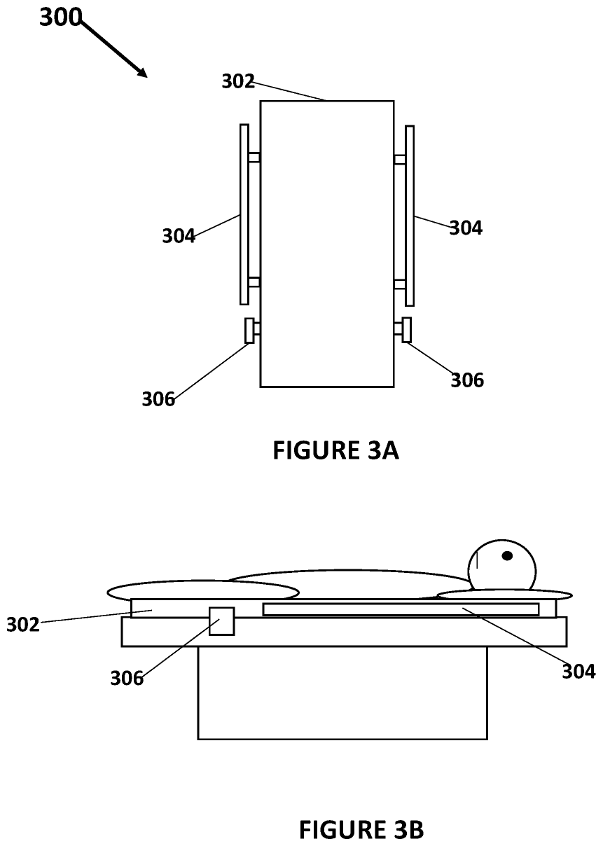

FIGS. 3A and 3B illustrates another example of a device 300 that is positioned on top of the table and the device on the table with a patient. The device 300 may have a board member 302 that may be slide underneath the patient and on top of the table with the patient as shown in FIG. 3B. The device 300, like the device 100 is portable in that it can be easily moved between different tables for different treatments. Note that the precise dimensions of the board member 302 (or 502 or 802 in other embodiments) may be varied depending on the size of the table. The device 300 may have a rail 304 on each side of the board 302, such as rail 304 along each long side of the board as shown in FIG. 3A. The device 300 may further have a clamp 306 adjacent each rail 304 that can be positioned near the feet of the patient as shown in FIG. 3B. Each clamp may be used to clamp the board 302 to the table.

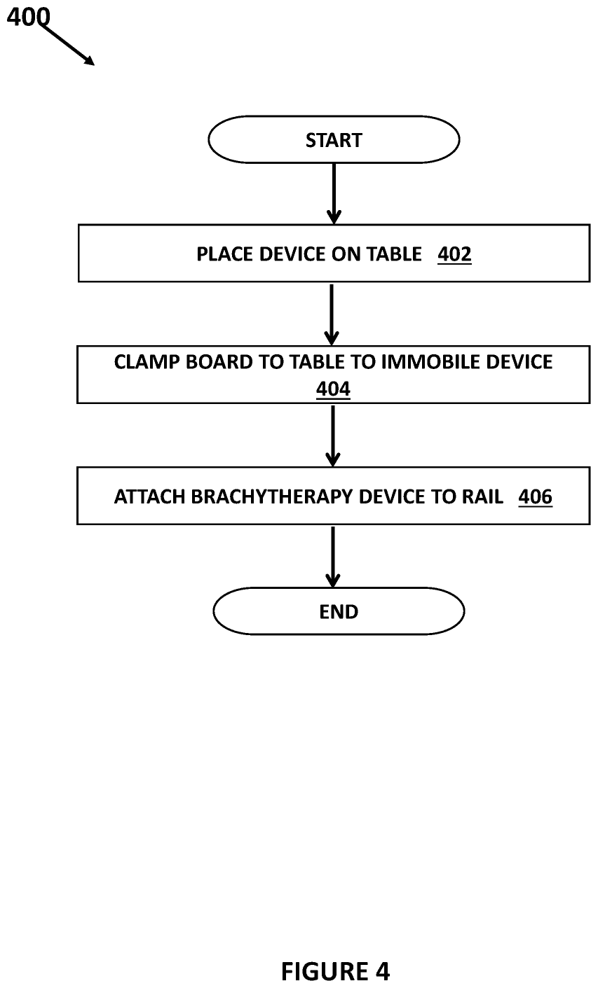

FIG. 4 illustrates another method 400 of using the device in FIGS. 3A and 3B with a brachytherapy device. The device 300 (402) may be placed onto the table and clamped to the table (404) using the clamps. Once the board is immobilized relative to the table, one or more treatment devices, such as one or more brachytherapy devices, may be attached to the rail (406) and thus fixed relative to the fixed frame of reference of the table since the treatment device is fixed to the rail that is fixed to the board that is fixed to the table.

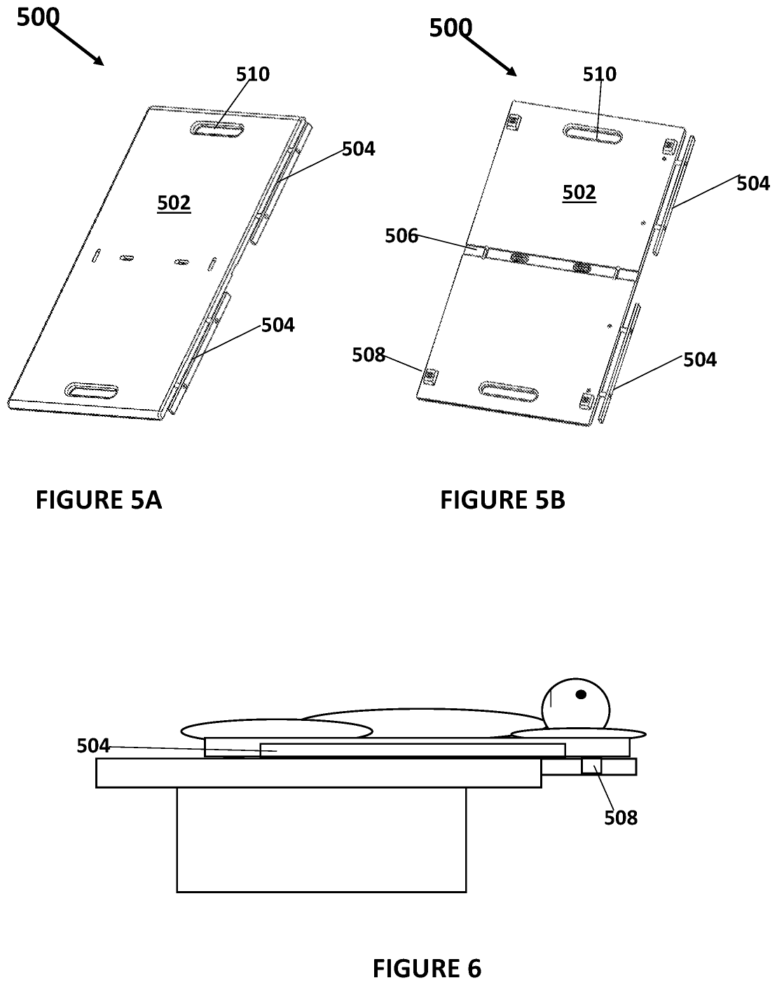

FIGS. 5A and 5B are a top view and a bottom view of another example of a device 500. In this embodiment, the device 500 may have a board member 502 that may slide underneath the patient and on top of the table with the patient as shown in FIG. 6. In this embodiment, the device 500 may one or more rails 504 that are both along the same long side of the board 502. The device 500 may further have a receiving feature 506 for fixation and a blocking feature 508 for fixation. The device 500 also may have one or more handles 510 that make it easier to carry and move the device 500.

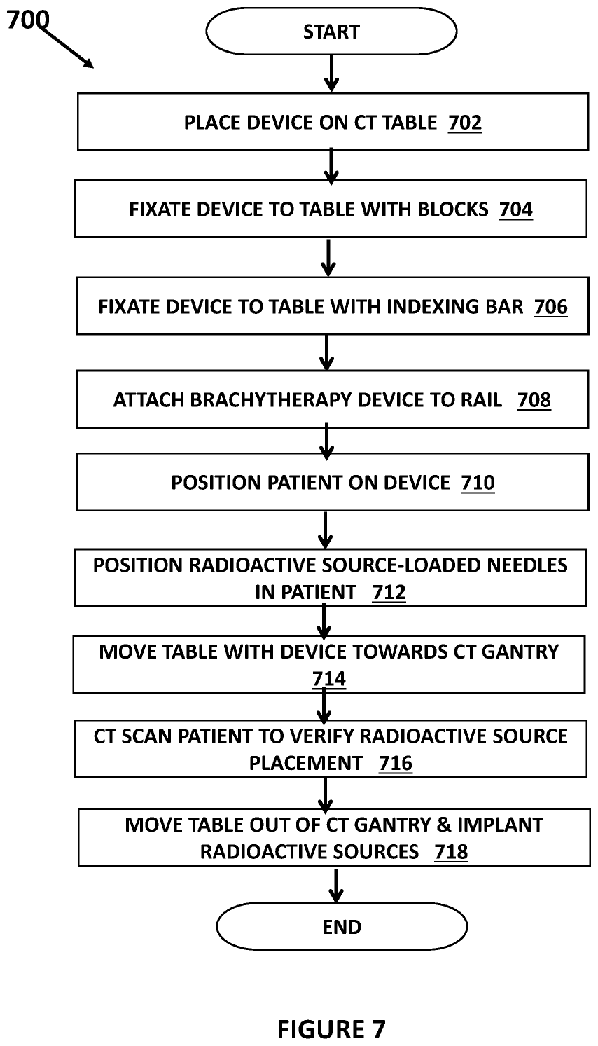

FIG. 7 illustrates another method 700 of using the device with a CT System. The device is placed on the CT table (702). The blocking features (blocks) may be used to fixate the device to the table (704). The receiving feature for an indexing bar, typically used with CT tables, also fixates the device on the table (706). The blocks prevent the device from sliding on the CT table. The indexing bar is attached to the CT table and has two pins that mate with the receiving feature on the device, which prevents the device from sliding on the CT table. Once the device is fixated relative to the CT table, one or more treatment devices, such as one or more brachytherapy devices, are attached to the rail(s) (708). The patient may then be positioned relative to the device (710). After the patient and brachytherapy device are positioned on the device, the clinician inserts radioactive source-loaded needles in the patient (712) using the brachytherapy device that may be a template through which the needle(s) are threaded. The CT table with the device and patient are moved towards the CT gantry for imaging (714). The clinician scans the patient to verify the positioning of the radioactive source applicator(s) (716). Once verified, the table and patient are moved away from the gantry and the clinician may implant or deliver the radioactive source(s) to the target treatment volume (718). The CT verification step is repeated as many times as necessary until the procedure is complete. In the embodiment used with CT scanning, the device may be manufactured out of a polymer-based material, stainless steel, stainless steel alloys, or any other suitable material. For example, polyethene and carbon fiber are compatible with CT and MRI. The device has indexing or reference features 506 that can be used with the imaging modality.

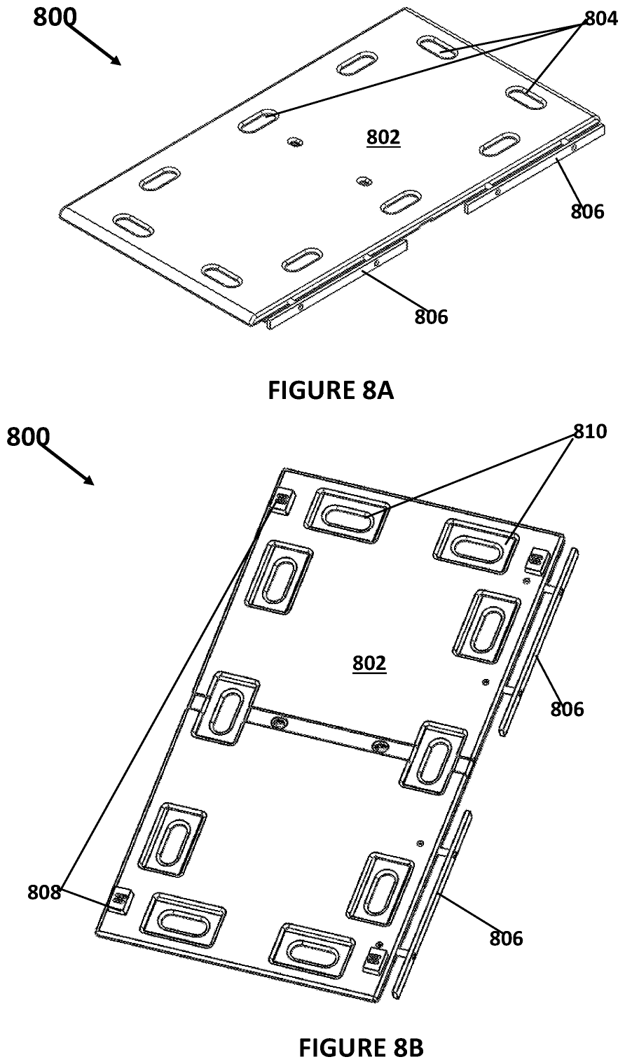

FIGS. 8A and 8B are a top view and a bottom view of another example of a device 800 with handles that have recessed pockets on the bottom side. In the embodiment, the device 800 has a board member 802 and one or more rails 806 along one long side of the board member 802. The device 800 may also have one or more handles 804 and one or more blocks 808 on a side opposite from the front side of the board as shown in FIG. 8B. On the bottom side of the board member 802, each handle is in a recessed pocket 810. The recessed pocket allows the user to place their fingers underneath the board when they grab the handle.

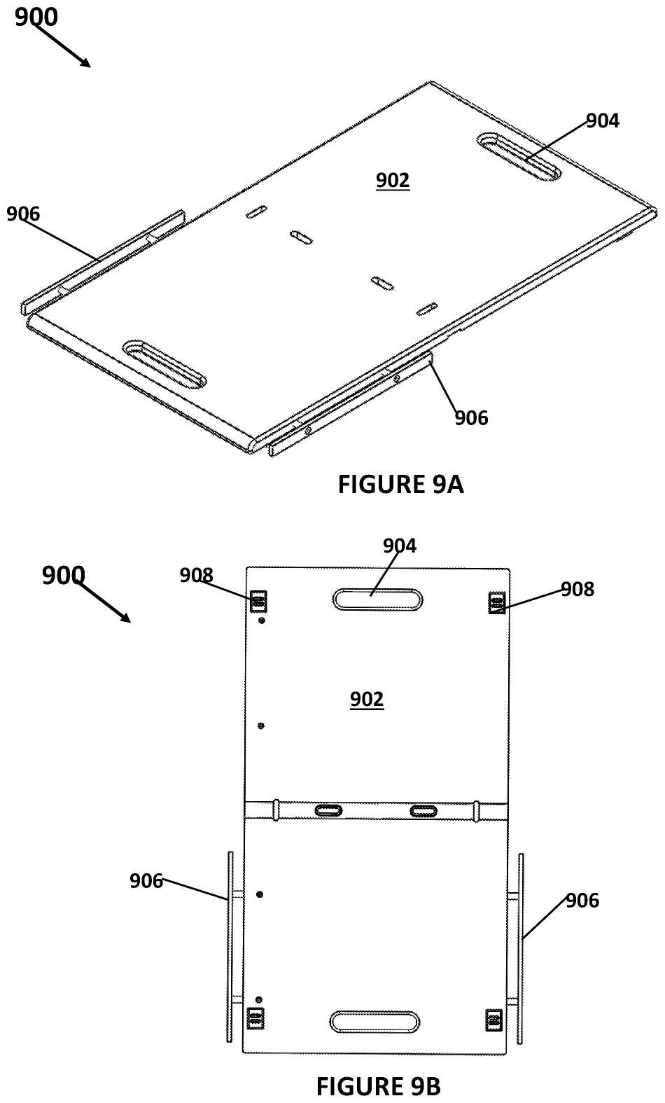

FIGS. 9A and 9B are a top view and a bottom view of another example of a device 900 with a rail 906 on opposite sides of a board member 902. In the example in FIGS. 9A and 9B, the rails 906 are adjacent to each other on the side portion of the board, but the rails could also be in other positions. Like some of the embodiments above, the device 900 may a set of blocks 908 and an indexing member near a centerline of the board member 902. Both of the embodiments in FIGS. 8A-9B can be used to perform a treatment like to device in FIGS. 5A and 5B.

Each of the above embodiments of the fixation support device may be a portable device that is capable of stabilizing and fixating one or more breast brachytherapy devices with an attachment mechanism at a position on or near a treatment surface, compatible with image guidance. The breast brachytherapy devices may be a brachytherapy template and armature and can have attachment mechanisms such as a rail clamp, pin, tightening knob, etc.

The portable device with one or more rails comprises one or a combination of the following: a main body that can be placed on top or near a treatment surface (e.g., a procedure table, CT table, Mill table, chair, etc . . . ); one or more wheels that can move the device wherein the one or more wheels can retract, the one or more wheels can lock in position, the one or more wheels are removable; and the device has one or more adjustable bases to lift the wheels away from the floor. The portable device may have one or more stabilizing mechanisms that can quickly and securely immobilize the device on a fixed frame of reference and One or more handles that can be used to move the device.

The device has the means to fixate one or more brachytherapy devices. Examples include but are not limited to: one or more bars that can be used to secure a brachytherapy device with a clamp; one or more clamps to secure a brachytherapy device; and a receiving feature to secure a brachytherapy device, such as a pin and slot mechanism to secure a brachytherapy device.

The device has the means to stabilize on a fixed or moving frame of reference (e.g., treatment surface—procedure table, CT table, MRI table, chair, etc . . . ). Examples may include but are not limited to: one side of the device has sufficient friction with the fixed frame of reference so that it is immobilized when a weight is placed on it; one or more clamps are used to fixate the device on the top, side, or bottom of the surface; one or more wheels on the body can retract or can be removed or can be lifted off the floor with an adjustable base, such that device is immobile; one or more protrusions are used to fixate the device on the surface; and/or one or more receiving features on the body that allows the use of the device with other brachytherapy fixation devices, such as an indexing bar.

The device has the means to be used in real-time or intermittently with imaging modalities such as CT, MRI, fluoroscopy, ultrasound, PET, and/or other novel medical imaging technology. Examples may include but are not limited to: the device may be manufactured out of a polymer-based material, stainless steel, stainless steel alloys, or any other suitable material. For example, polyethene and carbon fiber are compatible with CT and MRI; the device has indexing or reference features that can be used with the imaging modality.

The device may have a mechanism to move the rail along multiple axes. Examples include but are not limited to: a receiving feature for a rail so that the position of the rail can be adjusted and locked on the device; rack and pinion movement in a mechanical or hydraulic or pneumatic or power-assisted manner—the rail moves linearly or along one plane at a time; leadscrew-driven movement in a mechanical or hydraulic or pneumatic or power-assisted manner—the rail moves linearly or along one plane at a time; and/or motorized or robotic arm—the rail moves linearly and/or rotationally.

The foregoing description, for purpose of explanation, has been with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include and/or involve, inter alia, components such as software modules, general-purpose CPU, RAM, etc. found in general-purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general-purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software, and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure.

What is claimed is:

1. A device, comprising:
a portable device that is capable of stabilizing and fixating one or more breast brachytherapy devices;
an attachment mechanism connected to the portable device that is parallel to a treatment surface and is capable of being positioned on or near the treatment surface; and
wherein the device is compatible with image guidance.

2. The device of claim 1, wherein the attachment mechanism is a rail and the portable device is a base portion with an adjustable height.

3. The device of claim 2, wherein the base portion has one or more wheels that roll along a floor surface and an adjustment device to adjust a height of the rail off of the floor surface.

4. The device of claim 3, wherein the one or more wheels are retractable.

5. The device of claim 1, wherein the portable device is a board member, the attachment mechanism is a rail connected to each side of the board member and the portable device further comprises one or more clamps that are configured to clamp the board member to a treatment table.

6. The device of claim 1, wherein the portable device is a board member, the attachment mechanism is more than one rail with each rail connected to the same long side of the board member and wherein the board member further comprises one or more blocks and an indexing member that are configured to fixate the board to a treatment table.

7. The device of claim 1, wherein the portable device is a board member, the attachment mechanism is more than one rail with each rail connected to the same long side of the board member and wherein the board member further comprises one or more handles and a recessed pocket adjacent each handle.

8. The device of claim 1, wherein the portable device is a board member, the attachment mechanism is more than one rail with each rail connected to an opposite side of the board member and wherein the board member further comprises one or more handles and one or more blocks that are configured to fixate the board member to a treatment table.

9. A method for using a portable device, the method comprising:
moving the portable device adjacent to a treatment table;
immobilizing the portable device adjacent a treatment surface of the treatment table; and
fixating one or more breast brachytherapy devices adjacent the treatment surface using an attachment mechanism of the portable device that is parallel to the treatment surface.

10. The method of claim 9, wherein fixating the one or more breast brachytherapy devices further comprises using a rail and wherein the method further comprising adjusting a height of the rail relative to the treatment surface before immobilizing the portable device.

11. The method of claim 10, wherein moving the portable device further comprises rolling the portable device into a portion adjacent the treatment surface.

12. The method of claim 11, wherein immobilizing the portable device further comprises retracting a set of wheels.

13. The method of claim 9, wherein immobilizing the portable device further comprises clamping the portable device to the treatment table.

14. The method of claim 9 further comprises imaging a patient that is resting on the portable device.
